(19) United States
(12) Patent Application Publication
(10) Pub. No.: US 2025/0006295 A1
(43) Pub. Date: Jan. 2, 2025

(54) NEW DRUG CANDIDATE DISCOVERY SYSTEM AND COMPUTER PROGRAM IMPLEMENTING NEW DRUG CANDIDATE DISCOVERY PLATFORM

(71) Applicant: CALICI CO., LTD., Daejeon (KR)

(72) Inventors: Jae Mun CHOI, Daejeon (KR); Jin Hee PARK, Daejeon (KR); Jonathan WILLIANTO, Daejeon (KR); Van Huong LE, Daejeon (KR); Dinmukhamed MAILIBAY, Daejeon (KR); Nuzup SHADIEV, Daejeon (KR); Yu Kyung YUN, Daejeon (KR); Chul SUNG, White Plains, NY (US); Young Bin PARK, Seoul (KR)

(73) Assignee: CALICI CO., LTD., Daejeon (KR)

(21) Appl. No.: 18/276,808

(22) PCT Filed: Feb. 22, 2022
(86) PCT No.: PCT/KR2022/002602

§ 371 Date: Aug. 10, 2023

(30) Foreign Application Priority Data
Feb. 21, 2022 (KR) 10-2022-0022030

(51) Int. Cl. G16B 15/30

(52) U.S. Cl. CPC G16B 15/30

(57) ABSTRACT

Provided are a new drug candidate discovery system and a computer program implementing a new drug candidate discovery platform. A new drug candidate discovery system may include an automatic data preprocessing module configured to receive a target protein information from a user through a web interface, and perform preprocessing on a protein structure file obtained based on the target protein information; a simulation setting module configured to predict an Enzymatically Active Pocket for Docking Calculation (EAPDC) from the protein structure file using an artificial intelligence language model, and determine a docking calculation site; and a docking simulation module configured to perform a docking simulation for the docking calculation site.

### TECHNICAL FIELD

The present invention relates to a new drug candidate discovery system and a computer program implementing a new drug candidate discovery platform.

### BACKGROUND ART

Discovery of new drug candidates is the most time-consuming step in new drug development, and among various biotechnology research methods, computer-assisted in silico screening methods are attracting attention. In silico refers to computer simulations or computer programming in virtual experiments, and in silico screening refers to a search technology for candidate materials that is performed through a computer or computer simulation. In particular, in silico screening technology has recently been combined with big data analysis or artificial intelligence technology, and the scope of its application in the discovery and development of new drug candidates is gradually expanding.

However, in using the in silico screening method, general biologists need to collaborate with structural biologists or bioinformaticians to compute a ligand library containing a large amount of chemicals, or to collaborate with computer engineers in order to freely utilize big data analysis or artificial intelligence technology. Accordingly, there is a growing demand for an environment in which general biologists can fully utilize in silico screening by using only their own biological knowledge without the need to acquire special knowledge to use computing power.

### DISCLOSURE

### Technical Problem

One problem to be solved by the present invention is to provide a new drug candidate discovery system and a computer program implementing a new drug candidate discovery platform capable of supporting users with merely general biological knowledge to wholly engage in a drug candidate discovery process facilitated by in silico screening methods without acquiring knowledge in other fields.

### Technical Solution

A new drug candidate discovery system according to one aspect of the present disclosure, may include an automatic data preprocessing module configured to receive a target protein information from a user through a web interface, and perform preprocessing on a protein structure file obtained based on the target protein information; a simulation setting module configured to predict an Enzymatically Active Pocket for Docking Calculation (EAPDC) from the protein structure file using an artificial intelligence language model, and determine a docking calculation site; and a docking simulation module configured to perform a docking simulation for the docking calculation site.

The automatic data preprocessing module may obtain a protein structure file to be provided to the simulation setting module as a PDB (Protein Data Bank) file from a PDB database by receiving a PDB identifier from the user, or receive a PDB file directly from the user, the automatic data preprocessing module may detect and remove an Anisotropic B-factor from the PDB file, detect an Alternative Conformation from an amino acid residue field and correct the Alternative Conformation to an Non-Alternative Conformation, and detect Unusual Amino Acids in an amino acid residue field and modify the Unusual Amino Acids to non-specific amino acids corresponding to 20 species, when a missing residue is detected by examining a gap between residues in a protein structure of the PDB file, the automatic data preprocessing module obtains an appropriate protein amino acid sequence through a sequence database search, and automatically completes the missing residue in the protein amino acid sequence obtained.

The automatic data preprocessing module, when the protein structure file is not obtained from the PDB database or is not directly provided from the user, may obtain a protein structure file to be provided to the simulation setting module as a predicted structure modeled by inputting a protein amino acid sequence directly provided from the user into a protein structure prediction module.

The simulation setting module may set a rectangular box parameter to the predicted EAPDC, the new drug candidate discovery system may further include a user confirmation module for receiving confirmation of the rectangular box parameter from the user through the web interface.

The new drug candidate discovery system may further include a real-time notification module configured to, while the docking simulation is being performed, sort the predicted docking binding energies in real time to determine a ranking of candidate material, and provide the ranking of the candidate material to the user through the web interface.

The real-time notification module may provide a notification to the user in a method designated by the user when an event in which the ranking of the candidate material is changed occurs.

The new drug candidate discovery system may further include a verification request module configured to: convert the candidate material sorted by the real-time notification module into a 4D tensor form, re-predict the docking binding energy using a Convolutional Neural Network (CNN) and linear regression, and determine the ranking of the candidate material by reordering the candidate material according to the re-predicted docking binding energy.

The verification request module: may transmit a verification estimate request message for the candidate material selected by the user to a verification company server or a verification company account, receive a verification estimate message from the verification company server or the verification company account, and provide the verification estimate message to the user through the web interface, and transmit a verification request message to a server or an account of a verification company selected by the user through the web interface, wherein the verification request message includes at least one of at least one of requests for a synthesis of the candidate material, an enzyme inhibition experiment, a drug activity experiment, and a pharmacokinetic experiment.

A computer program that implements a platform for discovering new drug candidate from a target protein information, and stored on a computer-readable recording medium, the computer program may execute steps including: receiving the target protein information from a user through a web interface; obtaining a protein structure file based on the target protein information; performing preprocessing on the protein structure file; predicting an EAPDC from the protein structure file using an artificial intelligence language model and determining a docking calculation site; and performing a docking simulation for the docking calculation site.

The obtaining a protein structure file may include: obtaining a PDB file from a PDB database by receiving a PDB identifier from the user, or receiving a PDB file directly from the user, and the performing preprocessing on the protein structure file may include: detecting and removing an Anisotropic B-factor from the PDB file, detecting an Alternative Conformation from an amino acid residue field and correcting the Alternative Conformation to an Non-Alternative Conformation, detecting Unusual Amino Acids in an amino acid residue field and modifying the Unusual Amino Acids to non-specific amino acids corresponding to 20 species, when a missing residue is detected by examining a gap between residues in a protein structure of the PDB file, obtaining an appropriate protein amino acid sequence through a sequence database search, and automatically completing the missing residue in the protein amino acid sequence obtained.

The obtaining a protein structure file may include: when the protein structure file is not obtained from the PDB database or is not directly provided from the user, obtaining a protein structure file as a predicted structure modeled by inputting a protein amino acid sequence directly provided from the user into a protein structure prediction module.

The steps further including: setting a rectangular box parameter to the predicted EAPDC, and receiving confirmation of the rectangular box parameter from the user through the web interface.

The steps further including: while the docking simulation is being performed, sorting the predicted docking binding energies in real time to determine a ranking of candidate material, and providing the ranking of the candidate material to the user through the web interface, and providing a notification to the user in a method designated by the user when an event in which the ranking of the candidate material is changed occurs.

The steps further including: converting the candidate material sorted by the real-time notification module into a 4D tensor form, re-predicting the docking binding energy using a CNN and linear regression, and determining the ranking of the candidate material by reordering the candidate material according to the re-predicted docking binding energy.

The steps further including: transmitting a verification estimate request message for the candidate material selected by the user to a verification company server or a verification company account, receiving a verification estimate message from the verification company server or the verification company account, and providing the verification estimate message to the user through the web interface, and transmitting a verification request message to a server or an account of a verification company selected by the user through the web interface, wherein the verification request message includes at least one of at least one of requests for a synthesis of the candidate material, an enzyme inhibition experiment, a drug activity experiment, and a pharmacokinetic experiment.

### Advantageous Effects

According to the embodiments of the present invention, by automatically mitigating factors that may induce simulation errors within protein structure files, the precision and efficiency of drug candidate discovery can be enhanced, and the preprocessing of protein structure files can occur internally and automatically without the user's awareness and without requiring knowledge or collaboration with experts in other specialized fields so that an environment where the user can concentrate solely on drug candidate discovery can be provided.

Moreover, in the process of finding docking calculation sites on protein surfaces, it is implemented to easily utilize cloud server resources via a web interface, so that users are not required to learn complex Linux commands or collaborate with computer engineers to successfully find docking sites using artificial intelligence technology.

In addition, the user can not only monitor ranks of candidate materials in real time while the docking simulation is being performed, but also start verifying the ranked candidates even while the docking simulation is being performed without waiting for several months until the calculation of binding energies for all ligands is completed, and since the user can check the results in real time through a web browser, the entire process of new drug development can be conveniently monitored regardless of the type of user device, such as a smart phone, a tablet computer, or a desktop computer based on various operating systems.

Furthermore, users can easily request estimates and actual synthesis online to verify docking calculation results experimentally, not only can the time and cost required for analysis and synthesis of results be saved, but also the neutrality of the experimental results can be guaranteed by having the verification performed by a third party.

### DESCRIPTION OF THE DRAWINGS

FIG. 1 is a conceptual diagram illustrating a new drug candidate discovery system according to an embodiment of the present invention.

FIG. 2 is a block diagram illustrating a new drug candidate discovery system according to an embodiment of the present invention.

FIG. 3 is a flowchart illustrating a new drug candidate discovery method according to an embodiment of the present invention.

FIG. 4 illustrates an example of a web interface of a new drug candidate discovery system according to an embodiment of the present invention.

FIG. 5 illustrates an example of a web interface of a new drug candidate discovery system according to an embodiment of the present invention.

FIG. 6 and FIG. 7 illustrate an example method of searching for docking sites using an artificial intelligence language model according to an embodiment of the present invention.

FIG. 8 illustrates an example of a web interface of a new drug candidate discovery system according to an embodiment of the present invention.

FIG. 9 illustrates an example of a web interface of a new drug candidate discovery system according to an embodiment of the present invention.

FIG. 10 illustrates an example of 4D tensors and features related to reordering of candidate materials according to an embodiment of the present invention.

FIG. 11 illustrates an example of learning results of a CNN model associated with the reordering of candidate materials according to an embodiment of the present invention.

FIG. 12 illustrates an example of a web interface of a new drug candidate discovery system according to an embodiment of the present invention.

FIG. 13 is a block diagram illustrating a computing device according to an embodiment of the present invention.

### MODE FOR INVENTION

Hereinafter, embodiments of the present invention will be described in detail with reference to the accompanying drawings to enable easy understanding and implementation by those of ordinary skill in the art to which this invention pertains. However, the present invention may be implemented in many different forms and should not be construed as limited to the embodiments described herein. To clearly illustrate the invention, non-pertinent parts have been omitted from the drawings, and similar parts throughout the specification are marked with the similar reference numerals.

Throughout the specification and claims, when a part is described to “include” a certain component, it implies the inclusion of other components as well, not their exclusion, unless explicitly stated otherwise.

Furthermore, terms such as “part,” “unit,” “module,” and similar terms in the specification may denote a unit capable of performing at least one function or operation as described the present specification, and may be implemented through hardware, software, or a combination of both.

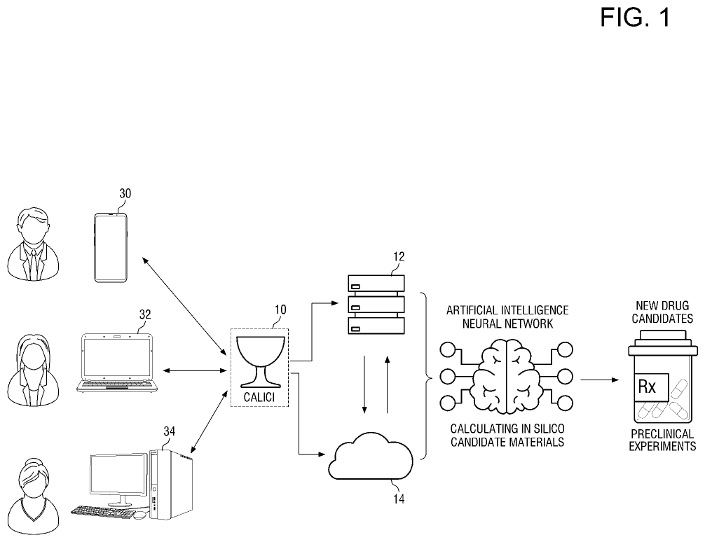

FIG. 1 is a conceptual diagram illustrating a new drug candidate discovery system according to an embodiment of the present invention.

Referring to FIG. 1, it is a diagram for explaining a new drug candidate discovery system 10 according to an embodiment of the present invention. The new drug candidate discovery system 10 can be implemented as a platform that provides users with services necessary for discovering new drug candidates in the form of a web service, and it may include any components in the form of programs, software, or hardware regardless of their form, if they are conceptually necessary for such implementation.

The new drug candidate discovery system 10 may provide the user devices 30, 32, and 34 with functions or services necessary for discovering new drug candidates. Specifically, the new drug candidate discovery system 10 provides various functions or services to the user devices 30, 32, and 34, such as enabling biologists with specific ideas for new drug development to utilize in silico screening method, without knowledge of other fields, to detect and remove errors in protein structure files, providing users with efficient detection of an Enzymatically Active Pocket for Docking Calculation (EAPDC) from protein structures, providing users with a real-time ranking of candidates based on docking binding energies while a lengthy docking simulation is performed; increasing reliability by predicting docking binding energy in two steps, enabling verification of candidate materials discovered through linkage with a verification company.

The new drug candidate discovery system 10, together with the new drug candidate discovery support server 12 and the cloud service support server 14, may calculate in silico candidate materials using an artificial intelligence neural network, and provide the user devices 30, 32, and 34 with various functions or services necessary for preclinical experiments on new drug candidates. Here, the new drug candidate discovery support server 12 may refer to a computing device capable of providing necessary data or executing commands in executing the platform, or a server instance running on the computing device, and operate in the back-end of the new drug candidate discovery system 10 implemented as a platform using a web service and provided as a front-end to user devices 30, 32, and 34. On the other hand, the cloud service support server 14 may be intended to provide an environment capable of processing big data analysis or computation using an artificial intelligence model on the cloud, it may refer to a computing device capable of providing various cloud services or a server instance running on the computing device.

The new drug candidate discovery system 10 operating as a platform at the front-end may provide the same function or service to user devices 30, 32, and 34 in various environments using a web interface. Specifically, for example, the first user device 30 may be a mobile device such as a smart phone or a tablet computer running a mobile operating system, the second user device 32 may be a notebook computer running a Windows operating system, and the third user device 34 may be a desktop computer running a Linux operating system. The new drug candidate discovery system 10, in the form of a platform implemented as a web service, allows user devices 30, 32, and 34 in different environments to calculate in silico candidate materials using an artificial intelligence neural network, and to equally use various functions or services necessary for preclinical experiments on new drug candidates, so that it has improved compatibility and user convenience, and has solved various problems requiring improvement in in silico calculations that have been previously performed through a terminal on Linux.

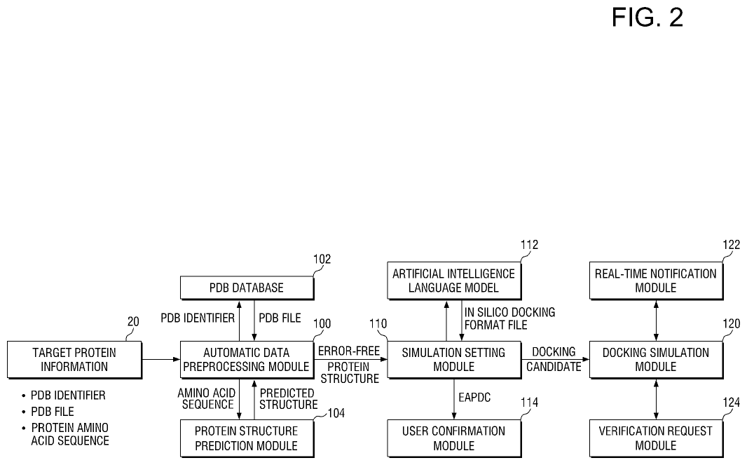

Hereinafter, with reference to FIG. 2 to FIG. 13, configurations and operating methods of the new drug candidate discovery system 10 according to embodiments of the present invention will be described in detail.

FIG. 2 is a block diagram illustrating a new drug candidate discovery system according to an embodiment of the present invention.

Referring to FIG. 2, the new drug candidate discovery system 10 according to an embodiment of the present invention may include an automatic data preprocessing module 100, a simulation setting module 110, and a docking simulation module 120.

The automatic data preprocessing module 100 may receive a target protein information 20 from a user through a web interface, and perform preprocessing on a protein structure file obtained based on the target protein information 20.

In discovering new drug candidates based on a protein structure, information on the target protein is required to discover new drug candidates that inhibit the target protein. However, in order to perform in silico screening method, detailed and accurate information on the target protein is required. Conventionally, a user had to directly search for or obtain detailed information on a target protein and manually input the detailed information, the user had to directly correct errors that were in the target protein structure file, and if a suitable structure was not found, a three-dimensional protein structure had to be modeled using a two-dimensional protein amino acid sequence, but these processes require understanding of protein structure, and were difficult to be solved alone by a general biologist, requiring collaboration with structural biologists or bioinformatics, and were costly and time-consuming. In addition, due to this, there was a problem that the accuracy of the in silico screening method was lowered or the failure rate of discovering candidate materials was high.

In order to improve this problem, the automatic data preprocessing module 100 introduced a method of preparing a target protein structure file used to determine docking binding sites in two ways. Specifically, the automatic data preprocessing module 100 adopts both a first method of receiving a PDB (Protein Data Bank) identifier from a user and acquiring a target protein structure file as a PDB file from the PDB database 102 or directly receiving a PDB file from a user, and a second method of inputting a protein amino acid sequence directly provided from a user into the protein structure prediction module 104 and obtaining a modeled predicted structure as a protein structure file. Here, the protein structure prediction module 104 may include a deep neural network model trained to predict a protein property from a protein amino acid sequence, predictable properties of proteins include the distance between amino acid pairs or angles between chemical bonds connecting amino acids, and the predicted structure may be output as a three-dimensional structure.

Regarding the first method, the PDB database 102 refers to an international public database as a ‘protein information bank’ that accumulates three-dimensional structures of biopolymers such as proteins. The automatic data preprocessing module 100 may obtain a PDB file by receiving a PDB identifier from a user and searching the PDB database 102, or directly acquire a PDB file from a user when the user has the PDB file.

However, the PDB file obtained in this way may contain factors that may cause errors in the subsequent docking simulation process, and the automatic data preprocessing module 100 may remove or correct the factors from the PDB file, thereby errors that may occur in the subsequent docking simulation process can be prevented in advance. Specifically, the automatic data preprocessing module 100 may detect and remove an Anisotropic B-factor from the PDB file, detect an Alternative Conformation from the amino acid residue field and correct it to an Non-Alternative Conformation, and detect Unusual Amino Acids in the amino acid residue field and modify them to non-specific amino acids corresponding to 20 species.

For example, if a docking simulation is performed without removing the Anisotropic B-factor from the PDB file, an error may occur in which the PDB file format is not recognized or the PDB file cannot be read, or if a docking simulation is performed with an Alternative Conformation or an Unusual Amino Acid present in the amino acid residue field, an error may occur due to an unknown amino acid, thereby accuracy in the performance of the in silico screening method may decrease or the failure rate of discovering candidate substances may increase. The automatic data preprocessing module 100 automatically handles such error occurrence factors, thereby preventing inefficiencies and inaccuracies that may occur when a user manually modifies a PDB file, and avoiding collaboration with a structural biologist, and the process of preprocessing the PDB file can be internally and automatically processed so that the user is not aware of it, providing an environment in which the user can focus only on discovering new drug candidates.

In addition, in relation to the first method, a correction may be performed for a missing residue in the protein structure of the PDB file. Specifically, the automatic data preprocessing module 100 may detect missing residues by examining gaps between residues in the protein structure of the PDB file, and when the missing residues are found, the automatic data preprocessing module 100 may obtain an appropriate protein amino acid sequence for completing the missing residue through a sequence database search, and automatically complete missing residues in the protein amino acid sequence obtained.

On the other hand, the second method of inputting a protein amino acid sequence directly provided from a user into the protein structure prediction module 104 and obtaining a modeled predicted structure as a protein structure file, may be performed secondarily when the first method fails, that is, when the automatic data preprocessing module 100 does not obtain the protein structure file from the PDB database 102 or not directly provided by the user. However, the scope of the present invention is not limited thereto, and the first method and the second method may be performed in parallel. For example, when the user provides all protein amino acid sequences together with the PDB identifier or the PDB file to the automatic data preprocessing module 100, the automatic data preprocessing module 100 may acquire both the protein structure file obtained by the first method and the protein structure file predicted by the second method, and the automatic data preprocessing module 100 may further increase the accuracy by displaying the results of the two methods to the user and confirming whether the target protein intended by the user is correct.

The simulation setting module 110 may receive an error-free protein structure file from which errors that may occur in the simulation are removed from the automatic data preprocessing module 100, and determine a docking calculation site by detecting an Enzymatically Active Pocket for Docking Calculation (EAPDC) from the protein structure file using the artificial intelligence language model 112.

Specifically, the simulation setting module 110 may predict a docking site (i.e., EAPDC) in the target protein structure using the artificial intelligence language model 112. Then, the simulation setting module 110 may set a rectangular box parameter to the predicted docking site and output it in an in silico docking file format.

To find the docking site in the target protein structure, a method in which the user calculates a rectangular box around the highest priority pocket and inputs to the docking program after calculating the Solvent Accessible Surface (SAS) of the protein surface, calculating the Center of Mass and the depth of the pocket from the surface, and giving rankings in order of the depth of the pocket, may be used. However, the pockets found by this method are often sites that do not affect the activity of the target protein at all, so there is a high probability of failing to derive a candidate material. On the other hand, there is also a method of analyzing sequences of similar amino acids with an alignment program and using the positional information of conserved residues together, but if the conserved sequences are conserved sequences for maintain protein folding (Conserved residues for Protein Folding and Structural Integrity), there is a concern that the analysis result will be ambiguous.

In order to derive effective drugs, inhibitors need to be designed in terms of enzymes rather than proteins. Accordingly, it is very important to classify each enzyme into a class and classify each gene according to the activity of the corresponding enzyme. There are EC_number and GO_number as classification systems for these enzymes. When a Graph Convolutional Network (GCN) is trained by implementing a protein's amino acid sequence and EC_number or GO_number as a Long short-term memory (LSTM) layer, EC_number or GO_number can be excellently predicted with only the amino acid sequence. However, although these EC_number and GO_number can help to understand the activity of an unknown enzyme, they cannot be directly utilized for in silico docking.

However, since amino acids that played an important role in classifying the enzyme are stored in a gradient class activity map for each enzyme class in each GCN layer, by extracting this and using it simultaneously with the pocket information calculated from SAS, it is possible to find an Enzymatic Active Pocket, i.e., EAPDC, without the interference of conserved sequences to maintain protein folding, thereby increasing the probability of finding candidates.

In order to find the EAPDC in the target protein structure and to improve the vulnerability of implementing a protein with a long amino acid sequence when implemented as an LTSM embedding layer, the simulation setting module 110 may implement a natural language processing model as an embedding layer and extract a gradient class activity map from the GCN layer learned with EC_number or GO_number. In addition, the simulation setting module 110 may find the EAPDC by combining the pocket values calculated from the SAS with the value of the gradient class activity map, and extract a box parameter required for docking.

In particular, when a natural language processing model is implemented as an embedding layer, when a missing residue exists in a target protein structure, an error may occur, making it difficult to predict the EAPDC. Therefore, as described above, the automatic data preprocessing module 100 may detect missing residues by examining gaps between residues in the target protein structure, and when the missing residues are found, obtain an appropriate protein amino acid sequence through a sequence database search and automatically complete missing residues in the obtained protein amino acid sequence, thereby preventing errors from occurring.

The user confirmation module 114 may display the rectangular box parameter for the predicted EAPDC through a web interface (or a web browser) to display it to the user and receive confirmation from the user. When the user's confirmation is completed, the predicted EAPDC may be determined as a docking calculation site and transmitted to the simulation setting module 110.

The docking simulation module 120 may perform a docking simulation for the docking calculation site determined by the simulation setting module 110. The docking simulation may be performed in a manner of examining stability of binding between the target protein and the candidate material, centering on the docking calculation site determined by the simulation setting module 110, to find a binding site with a stable energy state between the target protein and the candidate material. The docking simulation calculates docking through various chemical equations for a three-dimensional structure and calculates various complex equations to obtain information between a target protein and a candidate substance, and the amount of calculation is huge, and it takes a lot of time.

For example, using a Lamarckian Genetic Algorithm (LGA), it can output the expected binding energy in units of, for example, Kcal/mol while sequentially performing molecular docking on ligands provided from the ligand library. However, when using the LGA algorithm, since the binding energy is calculated for countless poses, that is, chemical conformations, exploring all possible poses requires a large amount of Central Processing Unit (CPU) time.

To reduce CPU time, docking based on the LGA algorithm can be performed by using a Compute Unified Device Architecture (CUDA) library to allocate the entire calculation in units of thread blocks on the GPU, and the search for the local pose can also improve efficiency and accuracy by searching for the most stable pose by applying a gradient descent algorithm. Nevertheless, the docking simulation is a process that takes several months depending on the size of the ligand library and the computing resources used, and a method for monitoring the progress is required. In addition, in the conventional method, after completing the calculation of the last ligand in the ligand library, a separate analysis process is required to verify the ligand with the highest binding energy, and there is also a need to improve this.

According to these requirements, while the docking simulation is performed by the docking simulation module 120, the real-time notification module 122 may sort the predicted docking binding energies from the highest binding energy in real time to determine the order of candidates and provide rankings of candidate materials to the user through a web interface. In addition, when an event in which the ranking of a candidate material is changed occurs while the docking simulation is being performed, the real-time notification module 122 may provide a notification to the user in a method designated by the user (e.g., email notification). Accordingly, the user can not only monitor the rankings of candidate materials in real time while the docking simulation is being performed, but also start verification of the ranked candidate materials even while docking simulation is being performed, without waiting for several months until the calculation of binding energies for all ligands is completed. In addition, since the user can check the results in real time through a web browser, the entire process of new drug development can be conveniently monitored regardless of the type of user device, such as a smartphone, a tablet computer, or a desktop computer based on various operating systems.

Meanwhile, the verification request module 124 may convert the candidate materials sorted by the real-time notification module 122 into a 4D tensor form and re-predict the docking binding energy using a Convolutional Neural Network (CNN) and linear regression and reorder the candidate materials according to the re-predicted docking binding energy to determine the order of the candidate materials. In this way, it is possible to discriminate more selected candidate materials, thereby increasing the prediction accuracy of the binding energy between the target protein and the candidate materials, compared to a method of binary classification of candidate materials by learning a CNN using the complex structure of a protein and a candidate material as an input value,

In particular, after the docking binding energy predicted by the real-time notification module 122 is sorted in real time from the highest binding energy to first determine the rankings of the candidate materials, for the docking candidate material generated therefrom, in the manner described above with respect to the verification request module 124, reliability of the candidate materials may be increased by re-predicting the docking binding energy and secondarily determining the rankings of the candidate materials by reordering the docking binding energy according to the re-predicted docking binding energy. Such a sorting result may be provided to the user through the web interface.

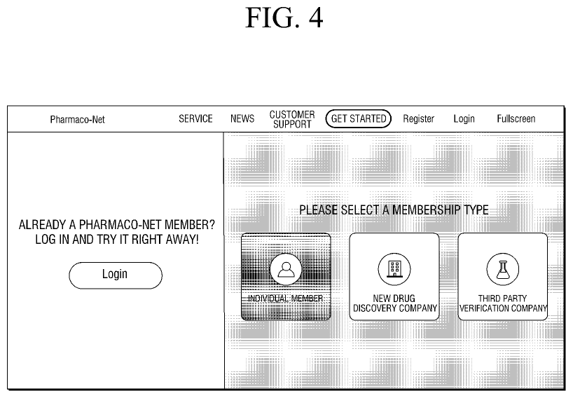

In addition, the verification request module 124 may transmit a verification estimate request message for the candidate material selected by the user to the verification company server through the web interface. To this end, the verification request module 124 may manage information on a plurality of verification companies using a database, and through an Application Programming Interface (API) provided from the verification company server or through a method of directly inquiring a third-party verification company that has joined the new drug candidate discovery system 10 to be described later in relation to FIG. 4 (for example, a method of sending a message to the verification company account), transmit a verification estimate request message to one or more verification company servers to inquire about the cost and duration of conducting the desired experiment on the candidate material selected by the user.

The verification request module 124 may receive verification estimate messages from one or more verification company servers, and provide a verification estimate message including an estimated cost and an estimated required period to the user through the web interface. When the user selects a preferred verification company on the web interface, the verification request module 124 may transmit a verification request message to the server of the verification company selected by the user. Here, the verification request message may include at least one of requests for a synthesis of candidate materials, a enzyme inhibition experiment, a drug activity experiment, and a pharmacokinetic experiment, and the scope of the present invention is not limited to the listed items, it may include any requests necessary for all stages from synthesis of candidate materials to development of new drugs. Accordingly, the user can easily request a estimation and actual synthesis online to verify the docking calculation result experimentally, thereby not only can the time and cost required for analysis and synthesis of results be saved, but also the neutrality of the experimental results can be guaranteed by having the verification performed by a third party.

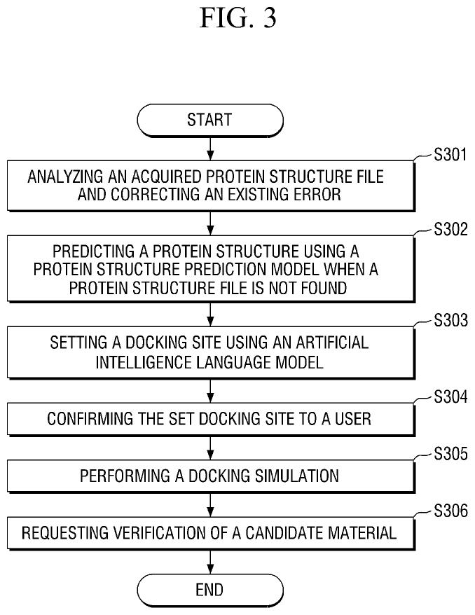

FIG. 3 is a flowchart illustrating a new drug candidate discovery method according to an embodiment of the present invention.

Referring to FIG. 3, a new drug candidate discovery method according to an embodiment of the present invention may include analyzing an acquired protein structure file and correcting an existing error S301. Specifically, S301 may include receiving a target protein information 20 from a user through a web interface; obtaining a protein structure file based on the target protein information 20; and performing preprocessing on the protein structure file.

Here, the obtaining of the protein structure file may include obtaining a PDB file from a PDB database by receiving a PDB identifier from a user, or receiving a PDB file directly from a user.

In addition, the performing of the preprocessing may include detecting and removing an Anisotropic B-factor from the PDB file; detecting an Alternative Conformation in the amino acid residue field and correcting it to an Non-Alternative Conformation; and detecting Unusual Amino Acids in the amino acid residue field and modifying them into non-specific amino acids corresponding to 20 species.

In addition, the performing the preprocessing may include obtaining an appropriate protein amino acid sequence through a sequence database search when a missing residue is detected by examining a gap between residues in the PDB file; and automatically completing missing residues from the obtained protein amino acid sequence.

In addition, the new drug candidate discovery method may include predicting a protein structure using a protein structure prediction model when a protein structure file is not found S302. Specifically, S302 may include, if the protein structure file has failed to be obtained from the PDB database 102 or not directly provided by the user, inputting the protein amino acid sequence directly provided from the user into the protein structure prediction module 104 and obtaining a modeled predicted structure as a protein structure file.

In addition, the new drug candidate discovery method may include setting a docking site using the artificial intelligence language model 112 S303. Specifically, S303 may include, predicting an EAPDC from a protein structure file using the artificial intelligence language model 112 and determining a docking calculation site.

In addition, the new drug candidate discovery method may include confirming the set docking site to a user S304. Specifically, S304 may include, setting a rectangle box parameter in the predicted EAPDC; and receiving confirmation of the rectangular box parameter from a user through a web interface.

Also, the new drug candidate discovery method may include performing a docking simulation on a docking calculation site S305.

In addition, the new drug candidate discovery method may include requesting verification of a candidate material S306. Specifically, S306 includes, transmitting a verification estimate request message for the candidate material selected by the user to the verification company server; receiving a verification estimate message from the verification company server and providing the verification estimate message to the user through a web interface; and transmitting a verification request message to the server of the verification company selected by the user through the web interface, and the verification request message may include at least one of requests for synthesis of candidate materials, an enzyme inhibition experiment, a drug activity experiment, and a pharmacokinetic experiment.

In some embodiments of the present invention, the new drug candidate discovery method may further include, while docking simulation is performed, sorting the predicted docking binding energies in real time to determine rankings of candidate materials and providing the rankings of candidate materials to a user through a web interface; and providing a notification to a user in a method designated by the user when an event in which the ranking of candidate materials is changed occurs.

Also, in some embodiments of the present invention, the new drug candidate discovery method may further include, converting the sorted candidate materials into a 4D tensor form; re-predicting docking binding energies using a CNN and linear regression; and reordering the candidate materials according to the re-predicted docking binding energy to determine the order of the candidate materials.

FIG. 4 illustrates an example of a web interface of a new drug candidate discovery system according to an embodiment of the present invention.

Referring to FIG. 4, as can be seen from the user sign-up screen of the web interface of the new drug candidate discovery system 10 according to an embodiment of the present invention, user types of the new drug candidate discovery system 10 may include individual members, new drug discovery companies, and third-party verification companies. Here, the individual member may be, for example, an individual (e.g., a general biologist) who wants to discover new drug candidates, and the new drug discovery company may refer to a company that wants to discover new drug candidates. The third-party verification company may refer to a verification company that the verification request module 124 intends to exchange with the verification company server a verification estimate request message, a verification estimate message, and a verification request message.

In this way, the new drug candidate discovery system 10 according to an embodiment of the present invention manages individual members and new drug discovery companies who wish to use the new drug candidate discovery function, and third-party verification companies as users, in order for manages individual members and new drug discovery companies to easily connect verification experiments on candidate materials derived through the new drug candidate discovery system 10.

A third-party verification company may receive a request for a verification estimate request or verification request directly from individual members and new drug discovery companies, and provide a reply to individual members and new drug discovery companies, or may use the API provided in the verification company server to provide link information that allows individual members and new drug discovery companies to send verification estimate request messages or verification request messages to the new drug candidate discovery system 10.

The new drug candidate discovery system 10 connects individual members and new drug discovery companies that want to use the new drug candidate discovery function, and a third-party verification company that can act as an agent for verification experiments on the derived candidates, thereby users can easily request estimates and actual synthesis online to verify docking calculation results experimentally, saving time and money required for result analysis and synthesis, and in addition, the neutrality of the experimental results can also be guaranteed by having the verification performed by a third party.

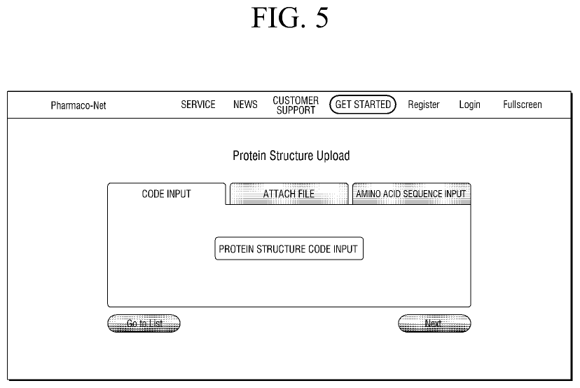

FIG. 5 illustrates an example of a web interface of a new drug candidate discovery system according to an embodiment of the present invention.

Referring to FIG. 5, as can be seen from the screen for receiving target protein information among the web interfaces of the new drug candidate discovery system 10 according to an embodiment of the present invention, the user may provide the PDB identifier to the new drug candidate discovery system 10 by inputting the PDB identifier into an input interface activated by selecting the “Code Input” tab. In addition, the user may directly provide the PDB file to the new drug candidate discovery system 10 by inputting the PDB file into an input interface activated by selecting the “Attach File” tab, or provide the protein amino acid sequence to the new drug candidate discovery system 10 by inputting the amino acid sequence to the input interface activated by selecting the “Input Amino Acid Sequence” tab.

In this way, the PDB identifier, the PDB file, or the protein amino acid sequence input by the user is transmitted to the automatic data preprocessing module 100 described above with reference to FIG. 2 to perform preprocessing on the protein structure file.

The new drug candidate discovery system 10 receives only target protein information from the user and internally processes a task of correcting factors that may cause errors during the simulation described above with reference to FIG. 2, so that the user can receive the result of the internally predicted docking site without performing any special operation just by inputting the target protein information, so that user convenience is guaranteed.

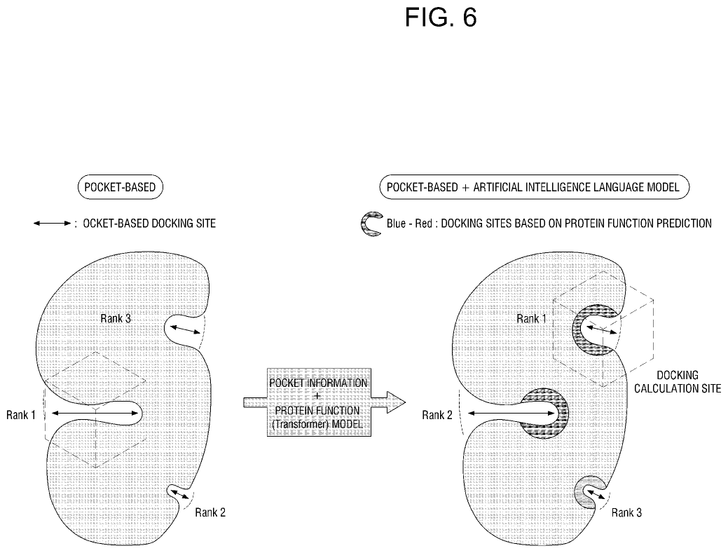

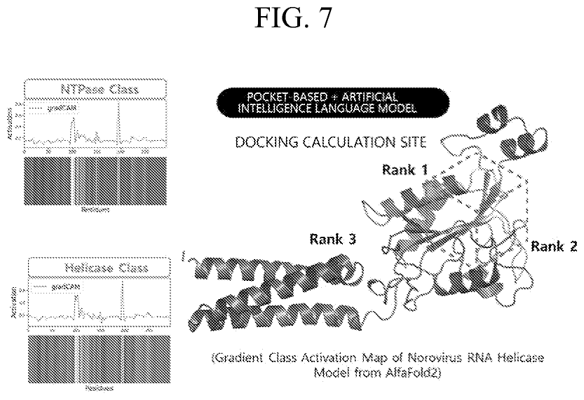

FIG. 6 and FIG. 7 illustrate an example method of searching for docking sites using an artificial intelligence language model according to an embodiment of the present invention.

Referring to FIG. 6, what is shown on the left shows a method of finding a pocket-based docking site by calculating the SAS of the protein surface and calculating the depth of the pocket, and what is shown on the right shows a method of finding a docking site based on protein function prediction by additionally applying the artificial intelligence language model 112 to the method of finding a pocket-based docking site. For example, in the method of finding a pocket-based docking site, the site marked “Rank 1” on the left was selected as a docking site because the depth of the pocket was the deepest, but the pocket may actually be a site that does not affect the activity of the target protein at all. In this case, if a site that does not affect the activity of a target protein is determined as a docking site, the probability of failing to derive a candidate material increases, therefore, according to the analysis result through the gradient class activity map using the artificial intelligence language model 112 according to an embodiment of the present invention, the docking site can be determined by considering which amino acids have a high contribution according to the activity level of the enzyme as well as the depth of the pocket. In this way, when finding a docking site based on protein function prediction, even if the site marked as “Rank 1” on the right side is not the deepest pocket, it is determined that it can greatly affect the activity of the target protein, and can be determined as a docking calculation site.

Next, referring to FIG. 7, when the user provides the amino acid sequence of the norovirus NTPase, and when the automatic data preprocessing module 100 does not obtain a PDB file from the PDB database 102, this is a case in which the predicted structure modeled by inputting the amino acid sequence of the norovirus NTPase directly provided from the user into the protein structure prediction module 104 is obtained as a protein structure file.

Accordingly, the simulation setting module 110 may predict the activity of Helicase based on the obtained protein structure file, generate a gradient class activity map for amino acids contributing to the activity prediction, and find a docking calculation site indicated as “Rank 1” by combining the values obtained from the gradient class activity map and the pocket values calculated from the SAS. Note that the site marked as “Rank 3” was predicted as the lowest ranked docking calculation site despite being the deepest pocket.

In this way, the docking calculation site is not determined simply by the depth of the pocket, but a site having a high effect on the activity of a target protein is determined as a docking calculation site in consideration of information on amino acids contributing to activity prediction, the probability of success in deriving a candidate substance can be increased.

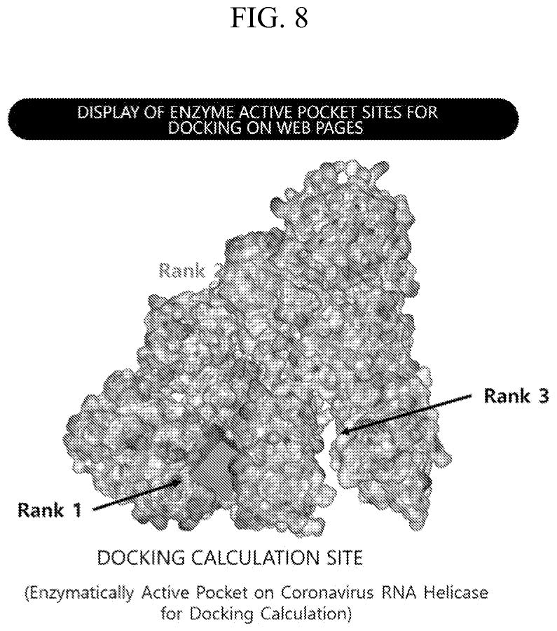

FIG. 8 illustrates an example of a web interface of a new drug candidate discovery system according to an embodiment of the present invention.

Referring to FIG. 8, as shown in the web interface of the new drug candidate discovery system 10 according to an embodiment of the present invention, by embedding the natural language processing model and rendering the rectangular box parameter for the EAPDC predicted using the GCN layer through a web interface (or web browser), it can be displayed to the user and confirmed by the user.

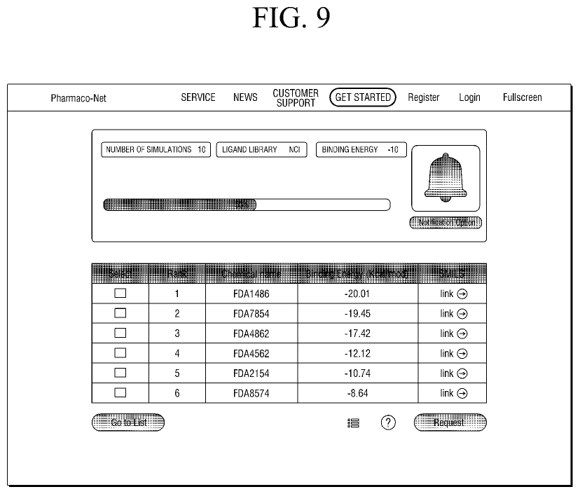

FIG. 9 illustrates an example of a web interface of a new drug candidate discovery system according to an embodiment of the present invention.

Referring to FIG. 9, as shown in the web interface of the new drug candidate discovery system 10 according to an embodiment of the present invention, while the docking simulation is performed by the docking simulation module 120, the real-time notification module 122 sorts the predicted docking binding energies from the highest binding energy in real time to provide a ranking of candidate materials to the user. Specifically, information such as rank, candidate material name, binding energy, and simplified molecular-input line-entry system (SMILES) code may be provided to the user. The users can monitor the ranking of candidate materials in real time through this web interface screen while docking simulation is performed, without waiting for several months until the calculation of binding energies for all ligands is completed, verification process for the desired candidate substance among the ranked candidates can be started with a simple method of checking the “Select” column and inputting the “Request” button even while docking simulation is being performed. In addition, since the user can check the results in real time through a web browser, the entire process of new drug development can be conveniently monitored regardless of the type of user device, such as a smartphone, a tablet computer, or a desktop computer based on various operating systems.

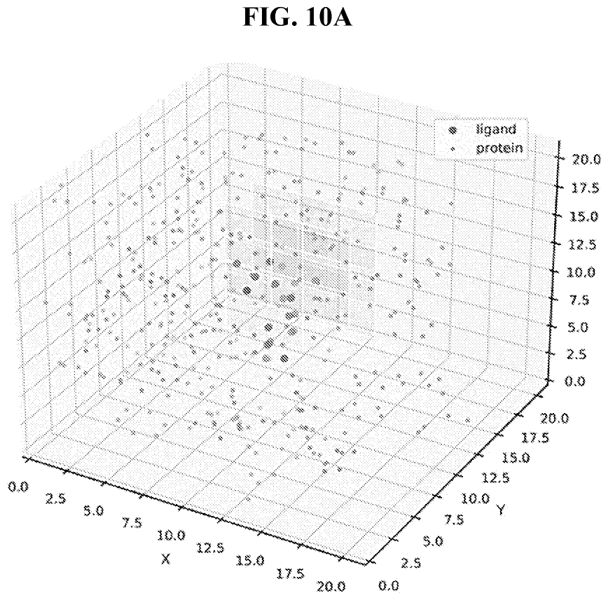

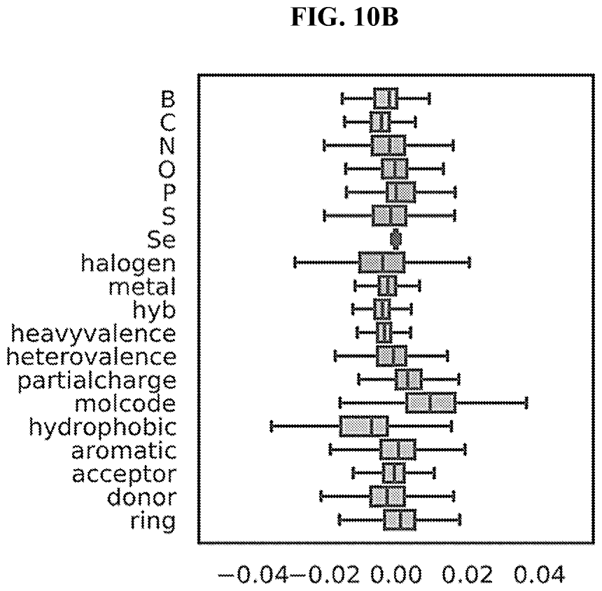

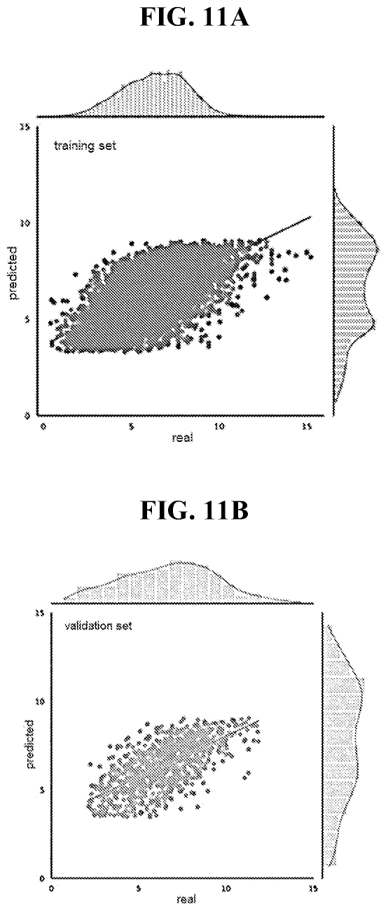

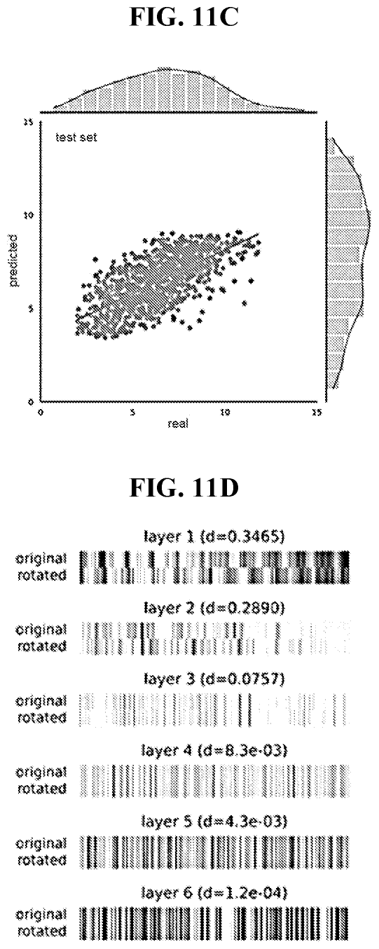

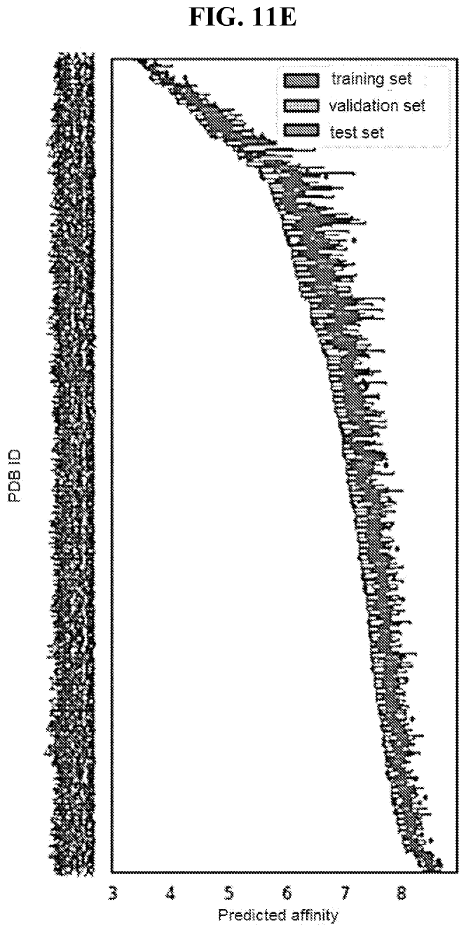

FIG. 10 illustrates an example of 4D tensors and features related to reordering of candidate materials according to an embodiment of the present invention, and FIG. 11 illustrates an example of learning results of a CNN model associated with the reordering of candidate materials according to an embodiment of the present invention.

Referring to FIG. 10, it shows that the verification request module 124 converted into a 4D tensor form to re-predict the docking binding energy using CNN and linear regression for the candidates sorted by the real-time notification module 122. “A)” shows an example of a 20 Å three-dimensional box enclosing a ligand binding site, and “B)” shows an example of a range of weights for 19 input features. In this way, it is possible to train a CNN by implementing a 20 Å 3D box and a 4D tensor including 19 input features, and each input feature can contribute to training the CNN.

Referring to FIG. 11, it shows the performance of the trained CNN, “A)”, “B)”, and “C)” denote a training set, a validation set, and a test set for binding energies, respectively, “D)” represents the activation of the hidden layer, and “E)” represents the predicted affinity. As shown in FIG. 11, even in the validation set and the test set, which were not actually learned, the difference between the predicted value and the actual value showed an average difference of 1.11−log K_{d} or −log Kᵢ. From this, the candidate materials sorted by the real-time notification module 122 can be converted into a form of 4D tensor, and the docking binding energy is re-predicted using CNN and linear regression, thereby it is possible to further increase the prediction accuracy of the binding energy between the target protein and the candidate material.

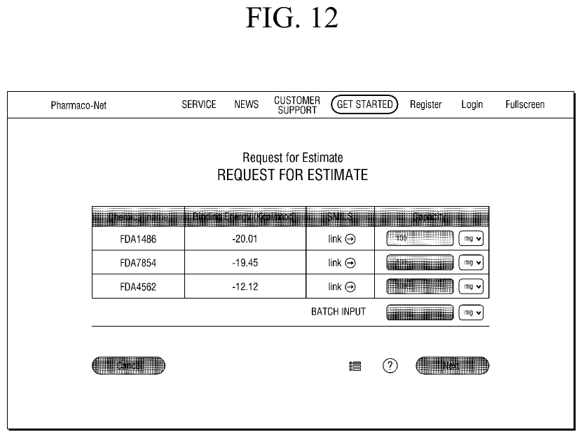

FIG. 12 illustrates an example of a web interface of a new drug candidate discovery system according to an embodiment of the present invention.

Referring to FIG. 12, as shown in the web interface of the new drug candidate discovery system 10 according to an embodiment of the present invention, the verification request module 124 may transmit a verification estimate request message for the candidate material selected by the user to the verification company server through the web interface. Specifically, cost negotiation and verification request can be realized with a simple method of entering the “Next” button by setting the volume desired for verification along with information such as candidate material name, binding energy, and SMILES code, through a verification company linked by the new drug candidate discovery system 10. As a result, efficiency in terms of time and cost can be achieved by removing the hassle of searching for verification companies one by one for users who want to discover new drug candidates, and the hassle of directly asking or requesting verification availability or cost. In addition, an advantageous effect arises in that the user can easily request necessary verifications in all stages from synthesis of candidate materials to new drug development.

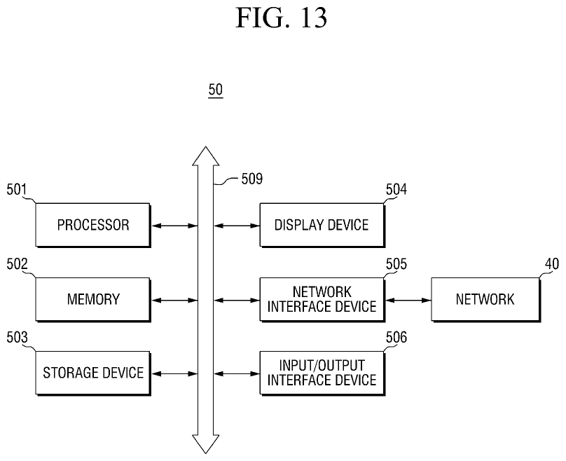

FIG. 13 is a block diagram illustrating a computing device according to an embodiment of the present invention.

Referring to FIG. 13, the new drug candidate discovery system, the new drug candidate discovery method, and the new drug candidate discovery platform according to embodiments of the present invention may be implemented using a computing device 50.

Computing device 50 includes at least one of a processor 501 communicating over a bus 509, a memory 502, a storage device 503, a display device 504, a network interface device 505 that provides a connection to the network 40 for communication with other entities, and an input/output interface device 506 providing a user input interface or a user output interface. Of course, although not shown in FIG. 13, the computer device 50 may further include any electronic device required to implement the technical ideas described herein.

The processor 501 may be implemented in various types such as an application processor (AP), a central processing unit (CPU), a graphic processing unit (GPU), a neural processing unit (NPU), and the like, and may be any electronic device that executes programs or instructions stored in the memory 502 or the storage device 503. In particular, the processor 501 may be configured to implement the functions or methods described above with reference to FIG. 1 to FIG. 12, and artificial intelligence-specific calculations in relation to the new drug candidate discovery system, the new drug candidate discovery method, and the new drug candidate discovery platform according to embodiments of the present invention may be processed on the GPU or the NPU.

The memory 502 and the storage device 503 may include various forms of volatile or non-volatile storage media. For example, the memory 502 may include read-only memory (ROM) or random access memory (RAM), and the memory 502 may be located internally or externally to the processor 501, and may be connected to the processor 501 through various known means. Meanwhile, examples of the storage device 503 include a hard disk drive (HDD) or a solid state drive (SSD), and the scope of the present invention is not limited to the elements listed above for description.

At least some of the new drug candidate discovery system, the new drug candidate discovery method, and the new drug candidate discovery platform according to embodiments of the present invention may be implemented as a program or software executed on the computing device 50, and such programs or software may be stored in a computer-readable medium.

Meanwhile, at least some of the new drug candidate discovery system, the new drug candidate discovery method, and the new drug candidate discovery platform according to embodiments of the present invention may be implemented using the hardware of the computing device 50 or may be implemented as separate hardware that may be electrically connected to the computing device 50.

According to the embodiments of the present invention described so far, by automatically mitigating factors that may induce simulation errors within protein structure files, the precision and efficiency of drug candidate discovery can be enhanced, and the preprocessing of protein structure files can occur internally and automatically without the user's awareness and without requiring knowledge or collaboration with experts in other specialized fields so that an environment where the user can concentrate solely on drug candidate discovery can be provided.

Moreover, in the process of finding docking calculation sites on protein surface, it is implemented to easily utilize cloud server resources via a web interface, so that users are not required to learn complex Linux commands or collaborate with computer engineers to successfully find docking sites using artificial intelligence technology.

In addition, the user can not only monitor rankings of candidate materials in real time while the docking simulation is being performed, but also start verifying the ranked candidates even while the docking simulation is being performed without waiting for several months until the calculation of binding energies for all ligands is completed, and since the user can check the results in real time through a web browser, the entire process of new drug development can be conveniently monitored regardless of the type of user device, such as smart phones, tablet computers, or desktop computers based on various operating systems.

Furthermore, users can easily request estimates and actual synthesis online to verify docking calculation results experimentally, not only can the time and cost required for analysis and synthesis of results be saved, but also the neutrality of the experimental results can be guaranteed by having the verification performed by a third person.

Although the embodiments of the present invention have been described in detail above, the scope of the present invention is not limited thereto, and various modifications and improvements made by those skilled in the art using the basic concept of the present invention defined in the following claims also fall within the scope of the present invention.

1. A new drug candidate discovery system, comprising:
an automatic data preprocessing module configured to receive a target protein information from a user through a web interface, and perform preprocessing on a protein structure file obtained based on the target protein information;
a simulation setting module configured to predict an Enzymatically Active Pocket for Docking Calculation (EAPDC) from the protein structure file using an artificial intelligence language model, and determine a docking calculation site; and
a docking simulation module configured to perform a docking simulation for the docking calculation site,
wherein the simulation setting module:
calculates a depth of a pocket based on Solvent Accessible Surface (SAS) of a surface of the target protein,
generates a gradient class activation map for an amino acid contributing to an activity prediction of the target protein, and
determines a site with a high effect on the activity of the target protein as a docking calculation site, considering the depth of the pocket, and the values for an amino acid with a high contribution in the gradient class activation map, and
wherein the gradient class activation map is extracted from a Graph Convolutional Network (GCN) trained using Enzyme Commission number (EC number) or Gene Ontology number (GO number) implemented as a natural language processing model as an embedding layer.

2. The new drug candidate discovery system of claim 1, wherein:
the automatic data preprocessing module obtains a protein structure file to be provided to the simulation setting module as a PDB (Protein Data Bank) file from a PDB database by receiving a PDB identifier from the user, or receives a PDB file directly from the user,
the automatic data preprocessing module detects and removes an Anisotropic B-factor from the PDB file, detects an Alternative Conformation from an amino acid residue field and corrects the Alternative Conformation to an Non-Alternative Conformation, and detects Unusual Amino Acids in an amino acid residue field and modifies the Unusual Amino Acids to non-specific amino acids corresponding to 20 species,
when a missing residue is detected by examining a gap between residues in a protein structure of the PDB file, the automatic data preprocessing module obtains a appropriate protein amino acid sequence through a sequence database search, and automatically completes the missing residue in the protein amino acid sequence obtained.

3. The new drug candidate discovery system of claim 1, wherein:
the automatic data preprocessing module, when the protein structure file is not obtained from a PDB database or is not directly provided from the user, obtains a protein structure file to be provided to the simulation setting module as a predicted structure modeled by inputting a protein amino acid sequence directly provided from the user into a protein structure prediction module.

4. The new drug candidate discovery system of claim 1, wherein:
the simulation setting module sets a rectangular box parameter to the predicted EAPDC,
the new drug candidate discovery system further comprises a user confirmation module for receiving confirmation of the rectangular box parameter from the user through the web interface.

5. The new drug candidate discovery system of claim 1, further comprising:
a real-time notification module configured to, while the docking simulation is being performed, sort the predicted docking binding energies in real time to determine a ranking of candidate material, and provide the ranking of the candidate material to the user through the web interface.

6. The new drug candidate discovery system of claim 5, wherein:
the real-time notification module provides a notification to the user in a method designated by the user when an event in which the ranking of the candidate material is changed occurs.

7. The new drug candidate discovery system of claim 5, further comprising:
a verification request module configured to:
convert the candidate material sorted by the real-time notification module into a 4D tensor form,
re-predict the docking binding energy using a Convolutional Neural Network (CNN) and linear regression, and
determine the ranking of the candidate material by reordering the candidate material according to the re-predicted docking binding energy.

8. The new drug candidate discovery system of claim 7, wherein:
the verification request module:
transmits a verification estimate request message for the candidate material selected by the user to a verification company server or a verification company account,
receives a verification estimate message from the verification company server or the verification company account, and provides the verification estimate message to the user through the web interface, and
transmits a verification request message to a server or an account of a verification company selected by the user through the web interface,
wherein the verification request message includes at least one of at least one of requests for a synthesis of the candidate material, an enzyme inhibition experiment, a drug activity experiment, and a pharmacokinetic experiment.

9. A computer program that implements a platform for discovering new drug candidate from a target protein information, and stored on a computer-readable recording medium, the computer program executes steps comprising:
receiving the target protein information from a user through a web interface;
obtaining a protein structure file based on the target protein information;
performing preprocessing on the protein structure file;
predicting an EAPDC from the protein structure file using an artificial intelligence language model and determining a docking calculation site; and
performing a docking simulation for the docking calculation site,
wherein the determining the docking calculation site includes:
calculating a depth of a pocket based on Solvent Accessible Surface (SAS) of a surface of the target protein,
generating a gradient class activation map for an amino acid contributing to an activity prediction of the target protein, and
determining a site with a high effect on the activity of the target protein as a docking calculation site, considering the depth of the pocket, and the values for an amino acid with a high contribution in the gradient class activation map, and
wherein the gradient class activation map is extracted from a Graph Convolutional Network (GCN) trained using Enzyme Commission number (EC number) or Gene Ontology number (GO number) implemented as a natural language processing model as an embedding layer.

10. The computer program of claim 9, wherein:
the obtaining a protein structure file includes:
obtaining a PDB file from a PDB database by receiving a PDB identifier from the user, or
receiving a PDB file directly from the user, and
the performing preprocessing on the protein structure file includes:
detecting and removing an Anisotropic B-factor from the PDB file,
detecting an Alternative Conformation from an amino acid residue field and correcting the Alternative Conformation to an Non-Alternative Conformation,
detecting Unusual Amino Acids in an amino acid residue field and modifying the Unusual Amino Acids to non-specific amino acids corresponding to 20 species,
when a missing residue is detected by examining a gap between residues in a protein structure of the PDB file, obtaining an appropriate protein amino acid sequence through a sequence database search, and
automatically completing the missing residue in the protein amino acid sequence obtained.

11. The computer program of claim 9, wherein:
the obtaining a protein structure file includes:
when the protein structure file is not obtained from a PDB database or is not directly provided from the user, obtaining a protein structure file as a predicted structure modeled by inputting a protein amino acid sequence directly provided from the user into a protein structure prediction module.

12. The computer program of claim 9, further executes steps comprising:
setting a rectangular box parameter to the predicted EAPDC, and
receiving confirmation of the rectangular box parameter from the user through the web interface.

13. The computer program of claim 9, further executes steps comprising:
while the docking simulation is being performed, sorting the predicted docking binding energies in real time to determine a ranking of candidate material, and providing the ranking of the candidate material to the user through the web interface, and
providing a notification to the user in a method designated by the user when an event in which the ranking of the candidate material is changed occurs.

14. The computer program of claim 13, further executes steps comprising:
converting the candidate material sorted by the real-time notification module into a 4D tensor form,
re-predicting the docking binding energy using a CNN and linear regression, and
determining the ranking of the candidate material by reordering the candidate material according to the re-predicted docking binding energy.

15. The computer program of claim 14, further executes steps comprising:
transmitting a verification estimate request message for the candidate material selected by the user to a verification company server or a verification company account,
receiving a verification estimate message from the verification company server or the verification company account, and providing the verification estimate message to the user through the web interface, and
transmitting a verification request message to a server or an account of a verification company selected by the user through the web interface,
wherein the verification request message includes at least one of at least one of requests for a synthesis of the candidate material, an enzyme inhibition experiment, a drug activity experiment, and a pharmacokinetic experiment.
